(19) United States
(12) Patent Application Publication
(10) Pub. No.: US 2021/0378808 A1
(43) Pub. Date: Dec. 9, 2021

(54) MALE URINARY INCONTINENCE STRIP

(71) Applicant: Jonathan K. Jensen, II, Cambridge, MA (US)

(72) Inventor: Jonathan K. Jensen, II, Cambridge, MA (US)

(21) Appl. No.: 17/027,345

(22) Filed: Sep. 21, 2020

Related U.S. Application Data
(60) Provisional application No. 63/036,815, filed on Jun. 9, 2020.

(51) Int. Cl. A61F 2/00; A61F 13/02

(52) U.S. Cl. CPC A61F 2/0009; A61F 13/025

(57) ABSTRACT

Disclosed herein is a strip for reducing involuntary urine leakage associated with male urinary incontinence. The strip comprises a first pad and a second pad. The strip also comprises a bridge that is configured to couple the first pad to the second pad. The strip additionally comprises an adhesive coupled to the first pad and the second pad. The first pad, the second pad, and the bridge are made of a non-resiliently flexible material.

### CROSS-REFERENCE TO RELATED APPLICATION

This application claims the benefit of U.S. Provisional Patent Application No. 63/036,815, filed Jun. 9, 2020, which is incorporated herein by reference.

### FIELD

The present disclosure relates generally to male urinary incontinence, and more particularly to devices and methods for reducing involuntary urine leakage associated with male urinary incontinence.

### BACKGROUND

Involuntary urine leakage due to male urinary incontinence is a common problem. According to some known techniques, urine leakage from the urethra is reduced by clamping or wrapping an external device onto the penis or using a device to constrict or plug the penis. However, conventional external devices are uncomfortable to wear and not particularly equipped to allow for voluntary urination while worn. Moreover, many external devices restrict blood flow to the penis when worn. Some conventional external devices are awkward and cumbersome when worn in conjunction with certain apparel, such as athletic apparel, or when used in conjunction with certain activities.

### SUMMARY

The subject matter of the present application has been developed in response to the present state of the art, and in particular, in response to the problems and needs of conventional devices and methods for addressing male urinary incontinence, that have not yet been fully solved. In view of the foregoing, the subject matter of the present application has been developed to provide apparatuses and methods for reducing involuntary leakage of urine, commonly associated with male urinary incontinence, that overcome many of the above-discussed shortcomings of the prior art. According to some examples, disclosed herein is a strip that is easy to put on, has an insubstantial size and streamlined shape, provides simple and quick access for voluntary urination, does not compromise blood flow to the penis to the extent associated with prior art devices, and helps to reduce involuntary urine leakage without excessive pressure on the penis.

Disclosed herein is a strip for reducing involuntary urine leakage associated with male urinary incontinence. The strip comprises a first pad and a second pad. The strip also comprises a bridge that is configured to couple the first pad to the second pad. The strip additionally comprises an adhesive coupled to the first pad and the second pad. The first pad, the second pad, and the bridge are made of a non-resiliently flexible material. The preceding subject matter of this paragraph characterizes example 1 of the present disclosure.

The bridge and the first pad form a one-piece construction. The second pad forms a construction that is separate from the bridge and the first pad. The bridge is manually non-permanently fastenable to the second pad to couple the first pad to the second pad. The preceding subject matter of this paragraph characterizes example 2 of the present disclosure, wherein example 2 also includes the subject matter according to example 1, above.

The strip further comprises a fastener fixed to one of the second pad or the bridge. The fastener is non-permanently fastenable to another one of the second pad or the bridge. The preceding subject matter of this paragraph characterizes example 3 of the present disclosure, wherein example 3 also includes the subject matter according to example 2, above.

The fastener is fixed to the bridge. The strip further comprises a second fastener fixed to the second pad. The fastener is non-permanently fastenable to the second fastener. The preceding subject matter of this paragraph characterizes example 4 of the present disclosure, wherein example 4 also includes the subject matter according to example 3, above.

The fastener comprises one of hooks or loops. The second fastener comprises another one of the hooks or loops. The hooks and the loops are engageable to non-permanently fasten together the fastener and the second fastener. The preceding subject matter of this paragraph characterizes example 5 of the present disclosure, wherein example 5 also includes the subject matter according to example 4, above.

The strip further comprises a first portion comprising the first pad and the bridge and a second portion comprising the second pad. The adhesive and the fastener are fixed to the same side of the first portion. The adhesive and the second fastener are fixed to opposite sides of the second portion. The preceding subject matter of this paragraph characterizes example 6 of the present disclosure, wherein example 6 also includes the subject matter according to any one of examples 4 or 5, above.

The adhesive comprises a first adhesive and a second adhesive. The first adhesive is coupled to the first pad. The second adhesive is coupled to the second pad. The first adhesive is spaced apart from the second adhesive by at least a gap along the bridge when the first pad is coupled to the second pad by the bridge. The fastener and the first adhesive are spaced apart by the gap. The preceding subject matter of this paragraph characterizes example 7 of the present disclosure, wherein example 7 also includes the subject matter according to example 6, above.

The bridge is narrower than at least one of the first pad and the second pad. The preceding subject matter of this paragraph characterizes example 8 of the present disclosure, wherein example 8 also includes the subject matter according to any one of examples 1-7, above.

The bridge is narrower than the first pad and the second pad. The preceding subject matter of this paragraph characterizes example 9 of the present disclosure, wherein example 9 also includes the subject matter according to any one of examples 1-8, above.

The adhesive is not coupled to at least a portion of the bridge at a location between the first pad and the second pad when the first pad is coupled to the second pad by the bridge. The preceding subject matter of this paragraph characterizes example 10 of the present disclosure, wherein example 10 also includes the subject matter according to any one of examples 1-9, above.

The strip further comprises a first tab coupled to and extending from the first pad, and

a second tab coupled to and extending from the second tab. The first tab has a smaller width than the first pad. The second tab has a smaller width than the second pad. The preceding subject matter of this paragraph characterizes example 11 of the present disclosure, wherein example 11 also includes the subject matter according to any one of examples 1-10, above.

The strip further comprises a stiffness member coupled to the bridge. The stiffness member has a stiffness that is greater than a stiffness of the bridge. The preceding subject matter of this paragraph characterizes example 12 of the present disclosure, wherein example 12 also includes the subject matter according to any one of examples 1-11, above.

The stiffness member is made of a material that is different than a material of the bridge. The preceding subject matter of this paragraph characterizes example 13 of the present disclosure, wherein example 13 also includes the subject matter according to example 12, above.

The adhesive comprises a first adhesive and a second adhesive. The first adhesive is coupled to the first pad. The second adhesive is coupled to the second pad. The first adhesive is spaced apart from the second adhesive by at least a gap along the bridge when the first pad is coupled to the second pad by the bridge. The preceding subject matter of this paragraph characterizes example 14 of the present disclosure, wherein example 14 also includes the subject matter according to any one of examples 1-13, above.

The first pad, the second pad, and the bridge form a one-piece monolithic construction. The preceding subject matter of this paragraph characterizes example 15 of the present disclosure, wherein example 15 also includes the subject matter according to example 14, above.

The first pad, the second pad, and the bridge form a one-piece monolithic construction. The bridge is wider than the first pad and the second pad. The adhesive spans from the first pad, across the bridge, to the second pad. An opening, across a portion of a width of the bridge, is formed in the adhesive. The preceding subject matter of this paragraph characterizes example 16 of the present disclosure, wherein example 16 also includes the subject matter according to any one of examples 10-15, above.

The first pad, the second pad, and the bridge collectively define an outer peripheral shape that is symmetrical. The preceding subject matter of this paragraph characterizes example 17 of the present disclosure, wherein example 17 also includes the subject matter according to any one of examples 1-16, above.

The first pad, the second pad, and the bridge collectively define an outer peripheral shape that is asymmetrical. The preceding subject matter of this paragraph characterizes example 18 of the present disclosure, wherein example 18 also includes the subject matter according to any one of examples 1-16, above.

Also disclosed herein is a method of regulating urine leakage from a urethral meatus of a glans penis. The method comprises adhering a first pad of a strip on a right side of the glans penis, adhering a second pad of the strip on a left side of the glans penis, extending a bridge of the strip across the urethral meatus from the first pad to the second pad, and tensioning the bridge to close the urethral meatus. The preceding subject matter of this paragraph characterizes example 19 of the present disclosure.

The step of tensioning the bridge comprises pulling the bridge relative to the second pad and non-permanently fastening the bridge to the second pad. The method further comprises releasing tension in the bridge, to open the urethral meatus, by unfastening the bridge from the second pad. The preceding subject matter of this paragraph characterizes example 20 of the present disclosure, wherein example 20 also includes the subject matter according to example 19, above.

The described features, structures, advantages, and/or characteristics of the subject matter of the present disclosure may be combined in any suitable manner in one or more examples and/or implementations. In the following description, numerous specific details are provided to impart a thorough understanding of examples of the subject matter of the present disclosure. One skilled in the relevant art will recognize that the subject matter of the present disclosure may be practiced without one or more of the specific features, details, components, materials, and/or methods of a particular example or implementation. In other instances, additional features and advantages may be recognized in certain examples and/or implementations that may not be present in all examples or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended numbered paragraphs, or may be learned by the practice of the subject matter as set forth hereinafter.

### BRIEF DESCRIPTION OF THE DRAWINGS

In order that the advantages of the subject matter may be more readily understood, a more particular description of the subject matter briefly described above will be rendered by reference to specific examples that are illustrated in the appended drawings. Understanding that these drawings, which are not necessarily drawn to scale, depict only certain examples of the subject matter and are not therefore to be considered to be limiting of its scope, the subject matter will be described and explained with additional specificity and detail through the use of the drawings, in which:

FIG. 1 is a schematic, perspective view of a strip for reducing involuntary urine leakage, according to one or more examples of the present disclosure;

FIG. 2 is a schematic, top plan view of the strip of FIG. 1, according to one or more examples of the present disclosure;

FIG. 3 is a schematic, bottom plan view of the strip of FIG. 1, shown with a first portion separated from a second portion, according to one or more examples of the present disclosure;

FIG. 4 is a schematic, side elevation view of the strip of FIG. 1, shown with a first portion being attached to a second portion, according to one or more examples of the present disclosure;

FIG. 5 is a schematic, side elevation view of the strip of FIG. 1, shown with a first portion attached to a second portion, according to one or more examples of the present disclosure;

FIG. 6 is a schematic, perspective view of a strip for reducing involuntary urine leakage coupled to a penis of a user, shown with a first portion unattached to a second portion, according to one or more examples of the present disclosure;

FIG. 7 is a schematic, perspective view of the strip of FIG. 6 coupled to the penis, shown with the first portion attached to the second portion, according to one or more examples of the present disclosure;

FIG. 8 is a schematic, top view of the strip of FIG. 6 coupled to the penis, shown with the first portion attached to the second portion, according to one or more examples of the present disclosure;

FIG. 9 is a schematic, side elevation view of a strip for reducing involuntary urine leakage coupled to a penis of a user, shown with a first portion unattached to a second portion, according to one or more examples of the present disclosure;

FIG. 10 is a schematic, side elevation view of the strip of FIG. 9 coupled to the penis, shown with the first portion attached to the second portion, according to one or more examples of the present disclosure;

FIG. 11 is a schematic, bottom plan view of a strip for reducing involuntary urine leakage, according to one or more examples of the present disclosure;

FIG. 12 is a schematic, bottom plan view of another strip for reducing involuntary urine leakage, according to one or more examples of the present disclosure;

FIG. 13 is a schematic, bottom plan view of yet another strip for reducing involuntary urine leakage, according to one or more examples of the present disclosure;

FIG. 14 is a schematic, bottom plan view of a strip for reducing involuntary urine leakage, according to one or more examples of the present disclosure;

FIG. 15 is a schematic, top plan view of a strip for reducing involuntary urine leakage, according to one or more examples of the present disclosure;

FIG. 16 is a schematic, side elevation view of the strip of FIG. 15, shown with a first portion attached to a second portion, according to one or more examples of the present disclosure; and

FIG. 17 is a schematic flow chart of a method of regulating urine leakage from a urethral meatus of a glans penis, according to one or more examples of the present disclosure.

### DETAILED DESCRIPTION

Reference throughout this specification to “one example,” “an example,” or similar language means that a particular feature, structure, or characteristic described in connection with the example is included in at least one example of the present disclosure. Appearances of the phrases “in one example,” “in an example,” and similar language throughout this specification may, but do not necessarily, all refer to the same example. Similarly, the use of the term “implementation” means an implementation having a particular feature, structure, or characteristic described in connection with one or more examples of the present disclosure, however, absent an express correlation to indicate otherwise, an implementation may be associated with one or more examples.

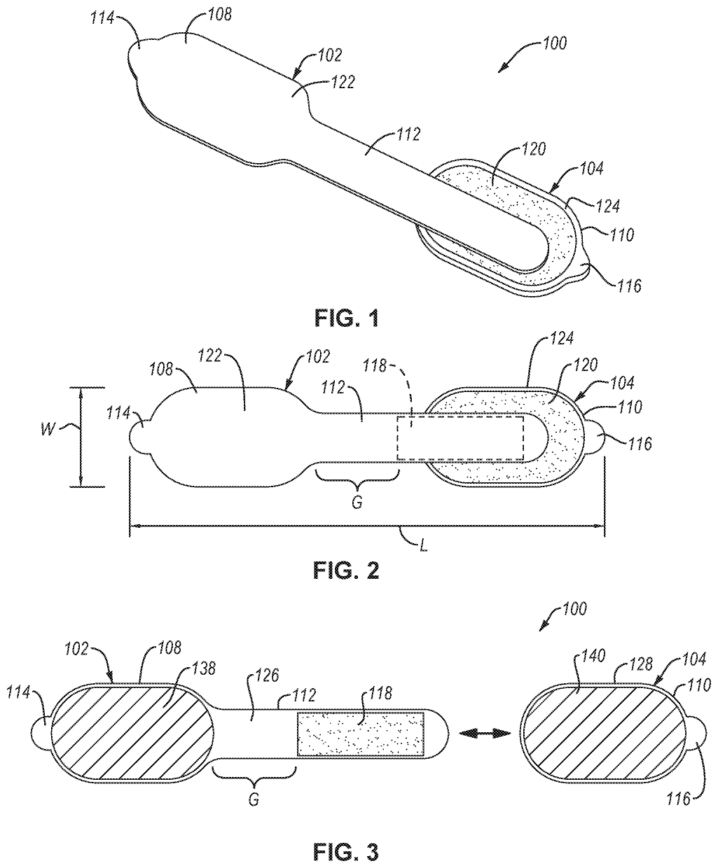

Referring to FIGS. 1-3, and according to some examples, disclosed herein is a strip 100 for reducing involuntary urine leakage associated with male urinary incontinence. The strip 100 includes a first portion 102 and a second portion 104. In the illustrated example, the first portion 102 and the second portion 104 are two separate and distinct units or pieces. In other words, the first portion 102 and the second portion 104 are separately formed and not permanently connected (e.g., form separate constructions). The first portion 102 includes a first pad 108 and the second portion 104 includes a second pad 110. The first portion 102 additionally includes a bridge 112 that is coupled to and extends from the first pad 108. The bridge 112 is co-formed with the first pad 108 in some examples to form a one-piece construction with the first pad 108. Additionally, in certain examples, the bridge 112 is narrower than the first pad 108 and the second pad 110 and thus acts as a neck between the first pad 108 and the second pad 110. In the illustrated example, the bridge 112 is longer than the first pad 108.

The first pad 108, the bridge 112, and the second pad 110 have a thin-walled construction. Accordingly, a width (W) and a length (L) of the first pad 108, the bridge 112, and the second pad 110 (see, e.g., FIG. 2) are substantially greater than a thickness (t) of the first pad 108, the bridge 112, and the second pad 110 (see, e.g., FIG. 5). The thickness of each one of the first pad 108, the bridge 112, and the second pad 110 is defined between a corresponding outer surface and inner surface of the first pad 108, the bridge 112, and the second pad 110. For example, the first pad 108 and the bridge 112 include a first outer surface 122 and a first inner surface 126, which defines a thickness of the first pad 108 and the bridge 112 therebetween. Likewise, the second pad 110 includes a second outer surface 124 and a second inner surface 128, which defines a thickness of the second pad 110 therebetween.

In some examples, the respective inner surfaces and the outer surfaces of the first pad 108, the bridge 112, and the second pad 110 are parallel to each other and flat. Accordingly, the first pad 108, the bridge 112, and the second pad 110 are sheet-like in certain examples. Moreover, in various examples, the first pad 108, the bridge 112, and the second pad 110 are flexible (e.g., non-resiliently flexible). In some examples, the first pad 108, the bridge 112, and the second pad 110 are elastic (e.g., stretchable). The first pad 108, the bridge 112, and the second pad 110 are made of the same material, in some examples, and different materials, in other examples. According to one example, the first pad 108, the bridge 112, and the second pad 110 are made of a polymeric material. In another example, the first pad 108, the bridge 112, and the second pad 110 are made of a fabric (e.g., a breathable fabric, a stretch fabric, a static fabric, a moisture-wicking fabric, a lotion-infused fabric, an ointment-infused fabric, and the like). The material of the first pad 108, the bridge 112, and the second pad 110 are made of a porous or semi-porous material in some examples to promote breathability.

Referring to FIG. 3, the first portion 102 further includes a first adhesive 138 coupled to the first inner surface 126 of the first pad 108. Similarly, the second portion 104 further includes a second adhesive 140 coupled to the second inner surface 128 of the second pad 110. The first adhesive 138 and the second adhesive 140 can be any of various adhesives know in the art. Generally, the first adhesive 138 and the second adhesive 140 are biocompatible tacky materials configured to adhere to the skin of the glans penis 132. Accordingly, the first adhesive 138 and the second adhesive 140 are tacky enough to temporarily adhere to the skin of the glans penis 132, but delicate enough not to irritate the skin of the glans penis 132, which can be more sensitive than the skin at most other areas of the body. In some examples, one or both of the first adhesive 138 and the second adhesive 140 is an adhesive-backed film or tape, such as Transpore™ cloth tape, Tegaderm Film™, or Mepitel Film™. As illustrated, in some examples, all or a substantial portion of the first inner surface 126 of the first pad 108 is covered by the first adhesive 138 and all or a substantial portion of the second inner surface 128 of the second pad 110 is covered by the second adhesive 140. The larger width or surface area of the first pad 108 and the second pad 110, relative to the bridge 112, promotes a larger contact area for the first adhesive 138 and the second adhesive 140 to adhere to the skin of the wearer.

The first portion 102 additionally includes a first tab 114 coupled to the first pad 108. Moreover, the second portion 104 also includes a second tab 116 coupled to the second pad 110. The first tab 114 is located at an opposite end of the strip 100 (e.g., when the first portion 102 is coupled to the second portion 104 as described below) than the second tab 116. The first adhesive 138 is not coupled to or does not cover the first tab 114 and the second adhesive 140 is not coupled to or does not cover the second tab 116. In some examples, the first tab 114 is defined by the first inner surface 126 and the first outer surface 122, and the second tab 116 is defined by the second inner surface 128 and the second outer surface 124. In some examples, the first tab 114 and the second tab 116 have smaller widths and smaller surface areas than the first pad 108 and the second pad 110, respectively.

The first portion 102 of the strip 100 additionally includes a first fastener 118 coupled (e.g., fixed) to the first inner surface 126 of the bridge 112. Accordingly, the first adhesive 138 and the first fastener 118 are fixed to the same side of the first portion 102. Correspondingly, the second portion 104 of the strip 100 further includes a second fastener 120 coupled (e.g., fixed) to the second outer surface 124 of the second pad 110. In other words, the second fastener 120 and the second adhesive 140 are on opposite sides of the second pad 100 or second portion 104.

The first fastener 118 extends along only a portion of the bridge 112 in some examples. For example, as shown in FIGS. 2 and 3, the first fastener 118 is located closer to a distal end of the bridge 112 than the first pad 108. More specifically, in some examples, the first fastener 118 is spaced apart from the first pad 108 (and the first adhesive 138 in certain examples) by a gap G. Accordingly, in these examples, the portion of the bridge 112 along the gap G is free of an adhesive as well as a fastener, which allows the bridge 112 to traverse the urethral meatus 134 without adhering to or abrading the glans penis 132 defining the urethral meatus 134. In other words, the adhesive is not coupled to at least a portion of the bridge 112 at a location between the first pad 108 and the second pad 110 when the first pad 108 is coupled to the second pad 110 by the bridge 112. However, in other examples, the first fastener 118 extends from approximately the first pad 108 such that no gap exists between the first pad 108 and the first fastener 118.

The second fastener 120 covers a substantial portion of the second outer surface 124 of the second pad 110. In certain examples, the second fastener 120 is wider than the first fastener 118 to allow for some flexibility in aligning the first fastener 118 relative to the second fastener 120.

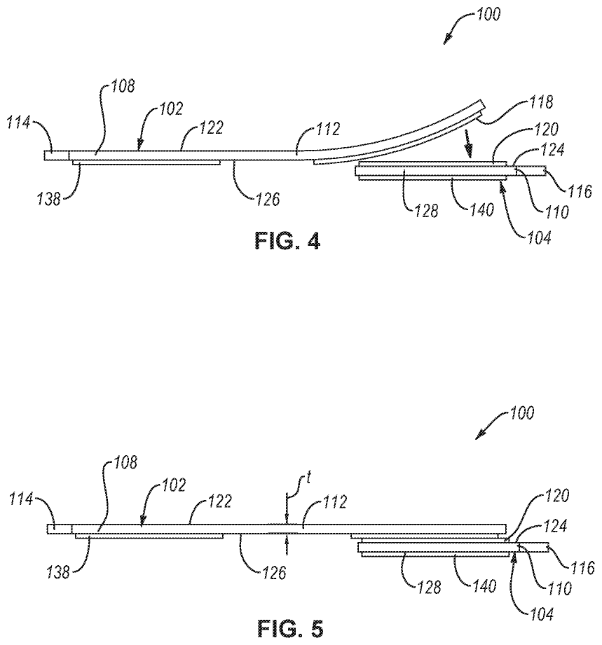

The first fastener 118 and the second fastener 120 are manually non-permanently fastenable to each other to intercouple the first portion 102 and the second portion 104 of the strip 100. Accordingly, the first fastener 118 and the second fastener 120 can be any of various fasteners configured to non-permanently fasten together. In one example, the first fastener 118 includes a plurality of hooks and the second fastener 120 includes a plurality of loops, such as associated with Velcro® fasteners. In contrast, according to another example, the first fastener 118 includes a plurality of loops and the second fastener 120 includes a plurality of hooks. The hooks engage the loops to create a non-permanent fastening of the first fastener 118 and the second fastener 120. More specifically, with referenced to FIGS. 4 and 5, the hooks and loops can be pressed together, to non-permanently fasten the hooks and loops together, and pulled apart with a predetermined force, to release the hooks from the loops. Although a hook-and-loop configuration provides certain advantages, in other examples, the first fastener 118 and the second fastener 120 are configured to non-permanently fasten together using other fastening structures and techniques. For example, the first fastener 118 and the second fastener 120 can be corresponding halves of a snap button, an interlocking mechanism, and the like. According to one example, the first fastener 118 and the second fastener 120 are adhesives that are selectively adhered together to facilitate the non-permanent fastening between them.

In some examples, the strip 100 includes only one of the first fastener 118 or the second fastener 120, which is manually non-permanently fastenable to the material of the opposing portion of the bridge 112 or the second pad 110 to intercouple the first portion 102 and the second portion 104. In other words, in some examples, the bridge 112 or the second pad 110 can be made from a fabric material that is configured to non-permanently attached to the one of the first fastener 118 or the second fastener 120. In this manner, the strip 100 only needs one fastener because the opposing fastener is integrated into the fabric material of the strip 100.

Referring to the examples of the strips of the present disclosure, according to one example with reference to the strip 100 (but applicable to examples of other strips), a method of using the strip 100 to help regulate (e.g., reduce) involuntary urine leakage includes adhering the first portion 102 of the strip 100 to a first side 142 of the glans penis 132 and adhering the second portion of the strip 100 to a second side 144 of the glans penis 132. The first portion 102 is adhered by pressing the first adhesive 138 on the first pad 108 onto the first side 142 of the glans penis 132. Similarly, the second portion 102 is adhered by pressing the second adhesive 140 on the second pad 110 onto the second side 144 of the glans penis 132. The first side 142 can be one of a right side or a left side of the glans penis 132 and the second side 144 can be the other of the right side or the left side of the glans penis 132. The left side of the glans penis 132 is the side portion of the glans penis 132 laterally left of the urethral meatus 134, or the sagittal plane 136, and the right side of the glans penis 132 is the side portion of the glans penis 132 laterally right of the urethral meatus 134, or the sagittal plane 136. Essentially, the sagittal plane 136 divides the wearer's body and penis 130 into the right side and the left side.

The first pad 108 is adhered to the first side 142 at a first distance from the urethral meatus 134 and the second pad 110 is adhered to the second side 144 at a second distance from the urethral meatus 134. As shown in FIG. 8, the first distance and second distance are selected such that the bridge 112 is able to extend across the urethral meatus 134 and the first fastener 118 is able to fasten to the second fastener 120.

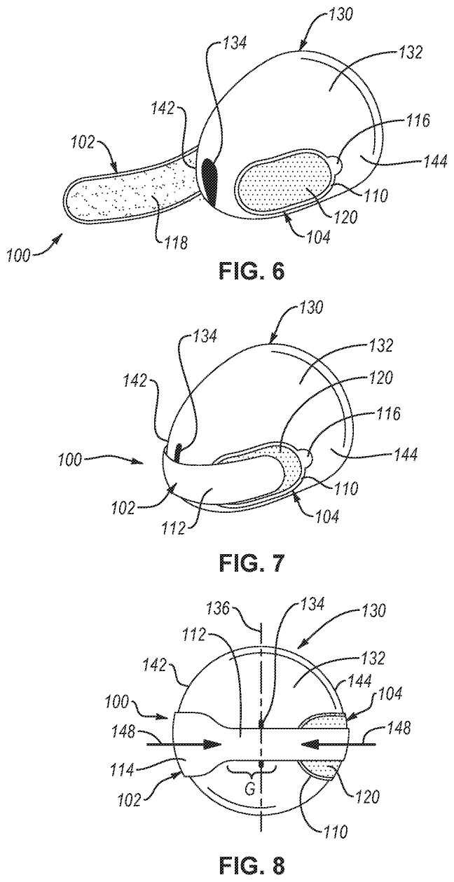

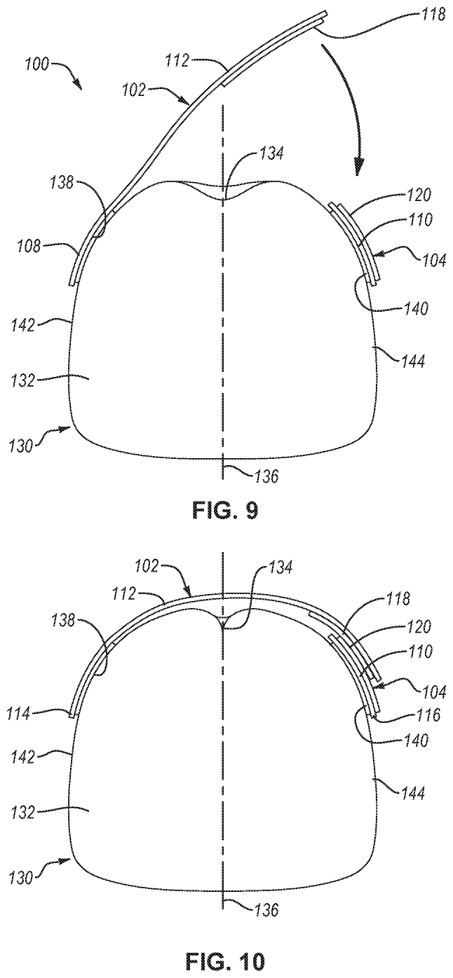

Accordingly, in some examples corresponding with the examples of FIGS. 6-10, the method of using the strip 100 further includes pulling the bridge 112 across the urethral meatus 134 and, with the bridge 112 in tension, fastening the first fastener 118 to the second fastener 120. The tension in the bridge 112 generates opposing inwardly-directed forces 148 (e.g., compressive forces (e.g., pinching) or medially-directed forces), perpendicular to the sagittal plane 136, on the tip of the glans penis 132. The inwardly-directed forces 148 cause the tissue of the glans penis 132 around the urethral meatus 134 to compress together, which closes the urethral meatus 134, across the width of the urethral meatus 134, as shown in FIGS. 7, 8, and 10. Accordingly, the adhesive itself does not close or seal the urethral meatus 134. Because the urethral meatus 134 is closed, involuntary urinary excretion through the urethral meatus 134 is prevented. Because the first portion 102 and the second portion 104 of the strip 100 are relatively thin, the strip 100 promotes non-intrusiveness and discreetness when the strip 100 is worn. Moreover, because the strip 100 is coupled to and overlays only a small portion of the glans penis 132, compared to existing products, the strip 100 promotes or reduces restriction of blood flow to the penis 130 when the strip 100 is worn.

While wearing the strip 100, should the wearer desire to voluntarily urinate, the wearer does not need to remove the strip 100. Rather, the first fastener 118 can be unfastened from the second fastener 120 to facilitate opening of the urethral meatus 134 to allow for voluntary urination. Referring to FIG. 6, with the first pad 108 and the second pad 110 retained on the glans penis 132, the wearer may unfasten the first fastener 118 from the second fastener 120 by grasping the bridge 112 and pulling the bridge 112 outwardly away from the glans penis 132 with a force greater than the fastening force between the first fastener 118 and the second fastener 120. Unfastening the first fastener 118 from the second fastener 118 releases the inwardly-directed forces 148, which releases the tension in the bridge 112 and the underlying tissue of the glans penis 132 around the urethral meatus 134. Without the tension in the tissue, the urethral meatus 134 is allowed to open to facilitate the voluntary discharge of urine from the urethral meatus 134. After voluntary urination is complete, the wearer can again pull the bridge 112, in tension, across the urethral meatus 134 and fasten the first fastener 118 to the second fastener 120 to reclose the urethral meatus 134 without removing the first pad 108 and the second pad 110 from the glans penis 132. In this manner, the wearer can voluntarily urinate without having to remove the strip 100.

However, when the wearer desires to remove the strip 100 from the glans penis 132, such as to replace the strip 100 with a new strip, the wearer can remove the first pad 108 and the second pad 110 from the glans penis 132. For example, the wearer can grasp the first tab 114 to pull and peel the first tab 114 away from the glans penis 132 and grasp the second tab 116 to pull and peel the second tab 116 away from the glans penis 132.

According to other examples, as shown in FIG. 11, disclosed herein is a strip 200 for reducing involuntary urine leakage associated with male urinary incontinence. Like the strip 100, the strip 200 functions to apply inwardly-directed forces on the glans penis 132 to close the urethral meatus 134. However, instead of the first portion 202 of the strip 200 being selectively detachable from the second portion 204 of the strip 200, the first portion 202 and the second portion 204 form a single unit or a one-piece monolithic construction. In other words, the bridge 212 is permanently coupled to both the first portion 202 and the second portion 204. Similar to the strip 100, the strip 200 includes a first pad 208 and a second pad 210, each having a corresponding one of a first adhesive 238 and a second adhesive 240 attached to an inner surface 252 of the corresponding one of the first pad 208 and the second pad 210. The bridge 212 is narrower than the first pad 108 and the second pad 110 and thus acts as a neck between the first pad 208 and the second pad 210. In some examples, the width of the bridge 212 is less than a length of the urethral meatus 134.

In some examples, a method of using the strip 200 to help reduce involuntary urine leakage includes adhering the first pad 208 of the strip 200 to the first side 142 of the glans penis 132 and adhering the second pad 210 of the strip 200 to the second side 144 of the glans penis 132. Like the strip 100, tension in the bridge 212 generates opposing inwardly-directed forces that cause the tissue of the glans penis 132 around the urethral meatus 134 to compress together, which closes the urethral meatus 134. However, instead of placing the bridge 212 in tension by pulling the bridge 212 and fastening the bridge to the second pad 210, the bridge 212 is placed in tension across the urethral meatus 134 by pulling the bridge 212 and the second pad 210, after the first pad 208 is adhered to the glans penis 134 on the first side 142, and, while the bridge 212 is in tension across the urethral meatus 143, adhering the second pad 210 to the glans penis 132 on the second side 144.

While wearing the strip 200, should the wearer desire to voluntarily urinate, the wearer does not need to entirely remove the strip 200. Rather, the first pad 208 or the second pad 210 can be decoupled from the glans penis 132 by pulling on the corresponding one of a first tab 214 or a second tab 216. Decoupling the first pad 208 or the second pad 210 from the glans penis 132 releases the inwardly-directed forces, which releases the tension in the bridge 212 and the underlying tissue of the glans penis 132 around the urethral meatus 134. Without the tension in the tissue, the urethral meatus 134 is allowed to open to facilitate the voluntary discharge of urine from the urethral meatus 134. After voluntary urination is complete, the wearer can again pull the bridge 112, in tension, across the urethral meatus 134 and re-adhere the first pad 208 or the second pad 210 to the glans penis 132 to reclose the urethral meatus 134. In this manner, the wearer can voluntarily urinate without having to completely remove the strip 200.

However, when the wearer desires to remove the strip 200 from the glans penis 132, such as to replace the strip 200 with a new strip, the wearer can remove the first pad 208 and the second pad 210 from the glans penis 132. For example, the wearer can grasp the first tab 214 to pull and peel the first tab 214 away from the glans penis 132 and grasp the second tab 216 to pull and peel the second tab 216 away from the glans penis 132.

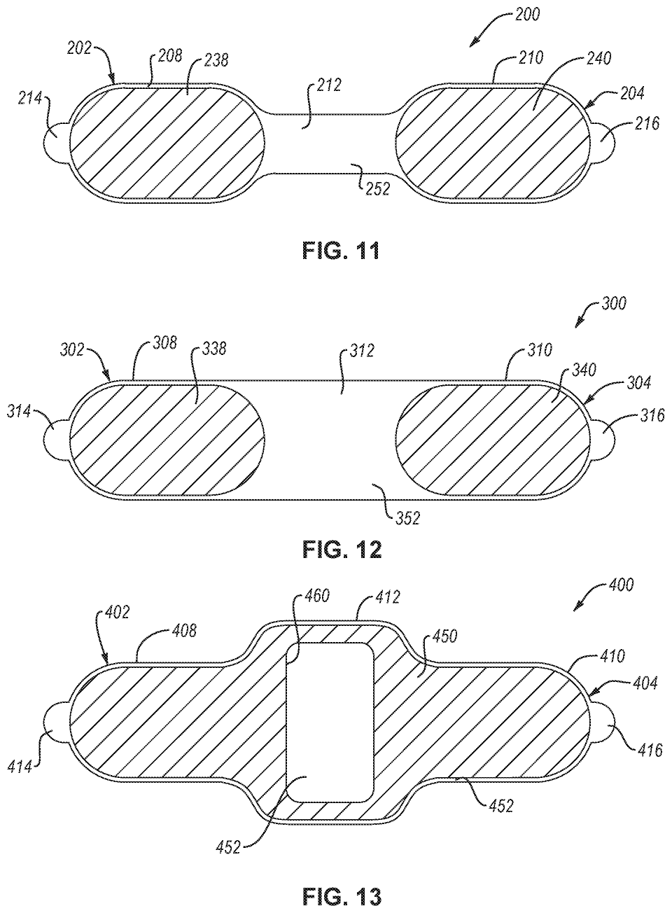

According to yet other examples, as shown in FIG. 12, disclosed herein is a strip 300 for reducing involuntary urine leakage associated with male urinary incontinence. Like the strip 200, the strip 300 is a one-piece unit that functions to apply inwardly-directed forces on the glans penis 132 to close the urethral meatus 134 by adhering a first adhesive 338 and a second adhesive 340, attached to an inner surface 352 of a corresponding one of a first pad 308 and a second pad 310 to the glans penis 132. However, instead of a bridge 312 of the strip 300 being narrower than the first pad 308 and the second pad 310 of the strip 300, a width of the bridge 312 is substantially that same as the widths of the first pad 308 and the second pad 310. Therefore, in some examples, the width of the bridge 212 is equal to or more than a length of the urethral meatus 134 such that an entirety of the urethral meatus 134 is covered by the bridge 212 when the strip 300 is worn. Such a configuration can help promote closure of the urethral meatus 134. It is also recognized that the bridge 112 of the strip 100 can have a width equal to or the same as the widths of the first pad 108 and the second pad 110 if desired.

According to another example, as shown in FIG. 13, disclosed herein is a strip 400 for reducing involuntary urine leakage associated with male urinary incontinence. Like the strip 200 and the strip 300, the strip 400 is a one-piece unit that functions to apply inwardly-directed forces on the glans penis 132 to close the urethral meatus 134 by adhering an adhesive 450 to the glans penis 132. However, instead of two separate adhesives associated with a first pad 408 and a second pad 410, the adhesive 450 is a single adhesive that spans from the first pad 408, along the bridge 412, to the second pad 410. Moreover, rather than being narrower than or equal to a width of the first pad 408 and the second pad 410, the bridge 412 is wider than the first pad 408 and the second pad 410. The bridge 412, being wider than the first pad 408 and the second pad 410, enables the adhesive 450 to extend continuously from the first pad 408 to the second pad 410. Additionally, the bridge 412, being wider, enables the addition of an exposed portion of an inner surface 452 through an opening 460 formed in the adhesive 450. The exposed portion is sized to cover the entire urethral meatus 134. In this manner, the adhesive 450 may adhere to the glans penis 132 around an entire periphery of the urethral meatus 134 without adhering to the urethral meatus 134. Such a configuration can help strengthen the adhesion of the strip 400 to the glans penis 132 and promote closure of the urethral meatus 134.

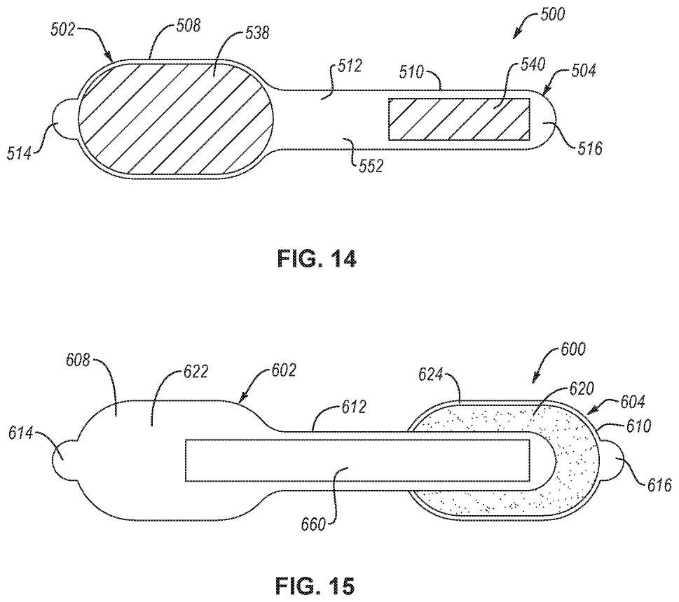

According to yet other examples, as shown in FIG. 14, disclosed herein is a strip 500 for reducing involuntary urine leakage associated with male urinary incontinence. Like the strip 200 and the strip 300, the strip 500 is a one-piece unit that functions to apply inwardly-directed forces on the glans penis 132 to close the urethral meatus 134 by adhering a first adhesive 538 and a second adhesive 540, attached to an inner surface 552 of a corresponding one of a first pad 508 and a second pad 510 to the glans penis 132. However, instead of an outer peripheral shape of the first pad, second pad, and bridge having a symmetrical shape, such as with the strip 100, the strip 200, and the strip 300, the outer peripheral shape of the first pad, the second pad, and the bridge of the strip 500 has an asymmetrical shape. That is, the first pad 508 has a shape and/or size that is different than the second pad 510. For example, as shown, the second pad 510 is narrower than the first pad 508. In some implementations, as shown, the second pad 510 has the same width as the bridge 512.

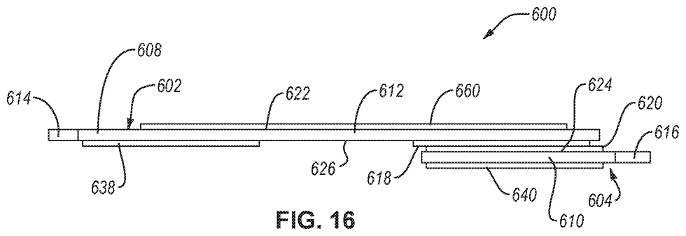

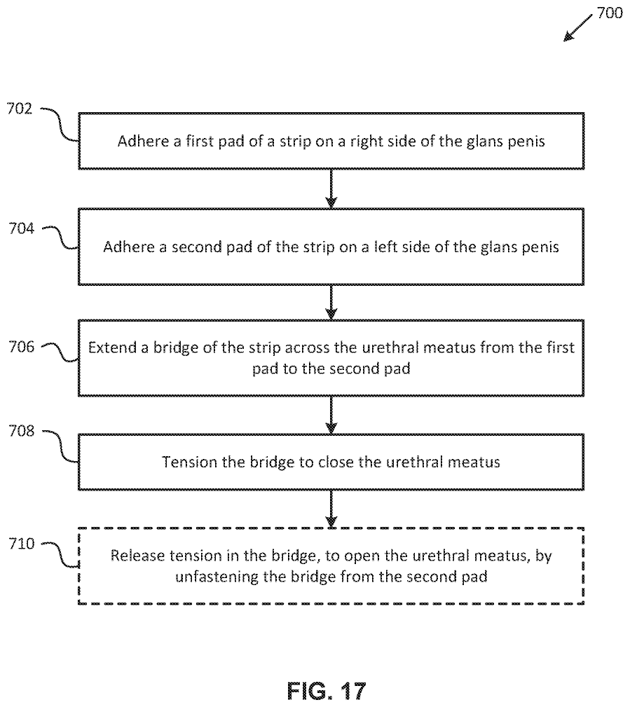

Referring to FIGS. 15 and 16, and according to other examples, disclosed herein is a strip 600 for reducing involuntary urine leakage associated with male urinary incontinence. Like the strip 100, the strip 600 includes a first portion 602 with a first pad 608 and first tab 614 and a second portion 604 with a second pad 610 and a second tab 616. Each one of the first pad 602 and the second pad 610 has a corresponding one of a first adhesive 638 and a second adhesive 640 (see, e.g., FIG. 16). The bridge 612 of the first portion 602 is narrower than the first pad 608 and the second pad 610 and thus acts as a neck between the first pad 608 and the second pad 610. The first portion 602 of the strip 600 additionally includes a first fastener 618 coupled to the first inner surface 626 of the bridge 612. Correspondingly, the second portion 604 of the strip 600 further includes a second fastener 620 coupled to the second outer surface 624 of the second pad 610. The first fastener 618 and the second fastener 620 are manually non-permanently fastenable to each other to intercouple the first portion 602 and the second portion 604 of the strip 600 in a manner similar to that of the sleeve 100.

However, unlike the strip 100, the first portion 602 of the strip 600 additionally includes a stiffness member 660 coupled to a first outer surface 622, which is opposite the first inner surface 626, of the bridge 612 and/or the first pad 608 such that the stiffness member 600 is co-movable (e.g., co-deformable) with movement (e.g., deformation) of the bridge 612 and/or the first pad 608. In some examples, the stiffness member 660 is coupled to and extends along the bridge 612 of the first portion 602. Accordingly, the stiffness member 660 can be elongated in a lengthwise direction that is parallel to the bridge 612.

The stiffness member 660 has a stiffness greater than the stiffness of the first portion 602. Accordingly, the stiffness member 660 adds stiffness to the first portion 602. Additionally, the stiffness member 660 is deformable (e.g., non-resiliently deformable) to promote a closing of the first portion 602 onto the second portion 604. Accordingly, in some examples, the stiffness member 660 helps maintain the shape of the first portion 602 (e.g., the bridge 612) when wrapped around the glans penis 132 and attached to the second portion 604. In certain examples, the stiffness member 660 is a thin strip, wire, or foil of a metallic material, or other similar material, that maintains a deformed shape following deformation. Accordingly, the stiffness member 660 is made of a material different than the first pad 608 and the bridge 612. The stiffness member 660 can be adhered to the first outer surface 622 of the first portion 602 or be integrated into or interposed between multiple layers of the first portion 602.

The above provides some examples of the strip of the present disclosure. In other examples, the strip can have any number of shapes and sizes and still accomplish the benefits presented above.

According to some examples, and referring to FIG. 16, a method 700 of using the strips of the present disclosure to regulate urine leakage from the urethral meatus 134 of the glans penis 132 includes (block 702) adhering the first pad of the strip on the right side 142 of the glans penis 132, (block 704) adhering the second pad of the strip on a left side 144 of the glans penis 132, (block 706) extending the bridge of the strip across the urethral meatus 134 from the first pad to the second pad, and (block 708) tensioning the bridge to close the urethral meatus 134. According to some examples, such as those using the strip 100, the step of tensioning the bridge at block 708 comprises pulling the bridge relative to the second pad and non-permanently fastening the bridge to the second pad. In such examples, the method 700 can further include (block 710) releasing tension in the bridge, to open the urethral meatus 134, by unfastening the bridge from the second pad.

In the above description, certain terms may be used such as “up,” “down,” “upper,” “lower,” “horizontal,” “vertical,” “left,” “right,” “over,” “under” and the like. These terms are used, where applicable, to provide some clarity of description when dealing with relative relationships. But these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise. Further, the term “plurality” can be defined as “at least two.” Moreover, unless otherwise noted, as defined herein a plurality of particular features does not necessarily mean every particular feature of an entire set or class of the particular features.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example, without limitation, two of item A, one of item B, and ten of item C; four of item B and seven of item C; or some other suitable combination.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

The present subject matter may be embodied in other specific forms without departing from its spirit or essential characteristics. The described examples are to be considered in all respects only as illustrative and not restrictive. All changes which come within the meaning and range of equivalency of the claims are to be embraced within their scope.

What is claimed is:

1. A strip for reducing involuntary urine leakage associated with male urinary incontinence, the strip comprising:
a first pad;
a second pad;
a bridge configured to couple the first pad to the second pad; and
an adhesive coupled to the first pad and the second pad;
wherein the first pad, the second pad, and the bridge are made of a non-resiliently flexible material.

2. The strip according to claim 1, wherein:
the bridge and the first pad form a one-piece construction;
the second pad forms a construction that is separate from the bridge and the first pad; and
the bridge is manually non-permanently fastenable to the second pad to couple the first pad to the second pad.

3. The strip according to claim 2, further comprising a fastener fixed to one of the second pad or the bridge, wherein the fastener is non-permanently fastenable to another one of the second pad or the bridge.

4. The strip according to claim 3, wherein:
the fastener is fixed to the bridge;
the strip further comprises a second fastener fixed to the second pad; and
the fastener is non-permanently fastenable to the second fastener.

5. The strip according to claim 4, wherein:
the fastener comprises one of hooks or loops;
the second fastener comprises another one of the hooks or loops; and
the hooks and the loops are engageable to non-permanently fasten together the fastener and the second fastener.

6. The strip according to claim 4, further comprising:
a first portion comprising the first pad and the bridge; and
a second portion comprising the second pad;
wherein:
the adhesive and the fastener are fixed to the same side of the first portion; and
the adhesive and the second fastener are fixed to opposite sides of the second portion.

7. The strip according to claim 6, wherein
the adhesive comprises a first adhesive and a second adhesive;
the first adhesive is coupled to the first pad;
the second adhesive is coupled to the second pad;
the first adhesive is spaced apart from the second adhesive by at least a gap along the bridge when the first pad is coupled to the second pad by the bridge; and
the fastener and the first adhesive are spaced apart by the gap.

8. The strip according to claim 1, wherein the bridge is narrower than at least one of the first pad and the second pad.

9. The strip according to claim 1, wherein the bridge is narrower than the first pad and the second pad.

10. The strip according to claim 1, wherein the adhesive is not coupled to at least a portion of the bridge at a location between the first pad and the second pad when the first pad is coupled to the second pad by the bridge.

11. The strip according to claim 1, further comprising:
a first tab coupled to and extending from the first pad; and
a second tab coupled to and extending from the second tab;
wherein:
the first tab has a smaller width than the first pad; and
the second tab has a smaller width than the second pad.

12. The strip according to claim 1, further comprising a stiffness member coupled to the bridge, wherein the stiffness member has a stiffness that is greater than a stiffness of the bridge.

13. The strip according to claim 12, wherein the stiffness member is made of a material that is different than a material of the bridge.

14. The strip according to claim 1, wherein:
the adhesive comprises a first adhesive and a second adhesive;
the first adhesive is coupled to the first pad;
the second adhesive is coupled to the second pad; and
the first adhesive is spaced apart from the second adhesive by at least a gap along the bridge when the first pad is coupled to the second pad by the bridge.

15. The strip according to claim 14, wherein the first pad, the second pad, and the bridge form a one-piece monolithic construction.

16. The strip according to claim 1, wherein:
the first pad, the second pad, and the bridge form a one-piece monolithic construction;
the bridge is wider than the first pad and the second pad;
the adhesive spans from the first pad, across the bridge, to the second pad; and
an opening, across a portion of a width of the bridge, is formed in the adhesive.

17. The strip according to claim 1, wherein the first pad, the second pad, and the bridge collectively define an outer peripheral shape that is symmetrical.

18. The strip according to claim 1, wherein the first pad, the second pad, and the bridge collectively define an outer peripheral shape that is asymmetrical.

19. A method of regulating urine leakage from a urethral meatus of a glans penis, the method comprising:
adhering a first pad of a strip on a right side of the glans penis;
adhering a second pad of the strip on a left side of the glans penis;
extending a bridge of the strip across the urethral meatus from the first pad to the second pad; and
tensioning the bridge to close the urethral meatus.

20. The method according to claim 19, wherein:
tensioning the bridge comprises pulling the bridge relative to the second pad and non-permanently fastening the bridge to the second pad; and
the method further comprises releasing tension in the bridge, to open the urethral meatus, by unfastening the bridge from the second pad.
